(19) United States
(12) Patent Application Publication
(10) Pub. No.: US 2026/0290530 A1
(43) Pub. Date: Sep. 24, 2026

(54) CONTENT GENERATION METHOD, ELECTRONIC DEVICE AND STORAGE MEDIUM

(71) Applicant: Beijing Zitiao Network Technology Co., Ltd., Beijing (CN)

(72) Inventors: Lingjie LUO, Beijing (CN); Wencheng ZHANG, Beijing (CN); Kangwei WU, Beijing (CN); Lei LIU, Beijing (CN); Shuping WANG, Beijing (CN); Meng LI, Beijing (CN); Jindong HONG, Hong Kong (CN); Yijun HE, Beijing (CN)

(21) Appl. No.: 19/576,887

(22) Filed: Mar. 24, 2026

(30) Foreign Application Priority Data
Mar. 24, 2025 (CN) 202510350110.9

(51) Int. Cl. G16H 15/00; G06F 40/279; G06F 40/35; G06F 40/40; G06T 11/00; G06V 10/764; G06V 30/412

(52) U.S. Cl. CPC G16H 15/00; G06F 40/279; G06F 40/35; G06F 40/40; G06T 11/00; G06V 10/764; G06V 30/412; G06V 2201/03; G06V 2201/134

(57) ABSTRACT

Embodiments of the present disclosure relate to a content generation method, an electronic device and a storage medium. The method includes: using a received image as a first image in response to an image uploading operation; obtaining respective field contents included in the first image in response to the first image belonging to a medical examination report; and generating a model prompt based on the respective field contents, and invoking a report interpretation model based on the model prompt to generate a report interpretation result of the first image, where the report interpretation model is obtained by fine-tuning a pre-trained generative model using medical examination report samples in advance. In this way, the generalization ability of the report interpretation model may be improved, and the flexibility of medical report interpretation and the readability and accuracy of medical report interpretation results may be improved.

### CROSS-REFERENCE TO RELATED APPLICATION

This application claims the priority to and benefits of the Chinese Patent Application, No. 202510350110.9, which was filed on Mar. 24, 2025. The aforementioned patent application is hereby incorporated by reference in its entirety.

### TECHNICAL FIELD

The embodiments of the present disclosure relate to a content generation method, an apparatus, an electronic device, a storage medium, and a program product.

### BACKGROUND

With the development of internet technologies, more and more users are accustomed to searching for medical-related information through the network. However, due to the high professionalism of medicine, the information that the internet can provide is limited. In particular, for understanding and searching of medical examination reports, it is difficult for the internet to give good search results. Based on this, relevant network services for interpreting the medical examination reports have emerged. Such services can perform character recognition and information extraction on medical report images uploaded by users, and understand and sort out the extracted information through certain medical rules (such as whether an indicator is beyond a normal range) or machine learning algorithms to generate interpretation results of medical examination reports.

However, the flexibility and adaptability of the above interpretation service for the medical examination reports are poor, and various medical examination reports cannot be interpreted well, resulting in one-sided, inaccurate and unreadable interpretation results.

### SUMMARY

In order to solve the above technical problems, embodiments of the present disclosure provide a content generation method, an apparatus, an electronic device, a storage medium, and a program product.

In a first aspect, an embodiment of the present disclosure provides a content generation method, the method includes:
using a received image as a first image in response to an image uploading operation;
obtaining respective field contents included in the first image in response to the first image belonging to a medical examination report; and
generating a model prompt based on the respective field contents, and invoking a report interpretation model based on the model prompt to generate a report interpretation result of the first image; where the report interpretation model is obtained by fine-tuning a pre-trained generative model using medical examination report samples in advance.

In a second aspect, an embodiment of the present disclosure further provides a content generation apparatus, and the apparatus includes:
a first image using module, configured to use a received image as a first image in response to an image uploading operation;
a field content obtaining module, configured to obtain respective field contents included in the first image in response to the first image belonging to a medical examination report; and
a report interpretation result generation module, configured to generate a model prompt based on the respective field contents, and invoke a report interpretation model based on the model prompt to generate a report interpretation result of the first image; where the report interpretation model is obtained by fine-tuning a pre-trained generative model using medical examination report samples in advance.

In a third aspect, an embodiment of the present disclosure further provides an electronic device, the electronic device includes:
a processor;
a memory, configured to store executable instructions; and
the processor is configured to read the executable instructions from the memory, and execute the executable instructions to implement the content generation method according to any one of the embodiments of the present disclosure.

In a fourth aspect, an embodiment of the present disclosure further provides a non-transitory computer-readable storage medium, the storage medium stores a computer program, the computer program, when executed by a processor, causes the processor to implement the content generation method according to any one of the embodiments of the present disclosure.

In a fifth aspect, an embodiment of the present disclosure further provides a computer program product, the computer program product is configured to perform the content generation method according to any one of the embodiments of the present disclosure.

According to the content generation method, the apparatus, the electronic device, the storage medium, and the program product provided by the embodiments of the present disclosure, a received image is used as a first image in response to an image uploading operation; respective field contents included in the first image are obtained in response to the first image belonging to a medical examination report; a model prompt is generated based on the respective field contents, and a report interpretation model, which is obtained by fine-tuning a pre-trained generative model using medical examination report samples in advance, is invoked based on the model prompt to generate a report interpretation result of the first image. In this way, the image of the medical examination report is interpreted by using the report interpretation model adapted to the medical field. On the one hand, the generalization ability of the report interpretation model is improved by using a widely-trained generative model with a certain scale, and the flexibility of medical report interpretation and the adaptability to various medical examination reports are improved without predefined rules or templates. On the other hand, the learning ability and context analysis ability of the report interpretation model are used to automatically capture complex non-linear relationships between multiple medical indicators, and generate more comprehensive, smooth and user-friendly medical report interpretation results by combining medical history, symptoms and examination data, thereby improving the readability and accuracy of the interpretation results, and improving the user experience.

It should be noted that the user information (including but not limited to user device information, user personal information, etc.) and data (including but not limited to data for analysis, stored data, displayed data, etc.) involved in the embodiments of the present disclosure are information and data authorized by the user or fully authorized by all parties, and the collection, use and processing of relevant data need to comply with relevant laws, regulations and standards of relevant countries and regions, and corresponding operation entrance is provided for the user to choose to authorize or reject.

### BRIEF DESCRIPTION OF DRAWINGS

In order to more clearly explain the technical solutions in the embodiments of the present disclosure, the drawings required to be used in the description of the embodiments will be briefly introduced below. Obviously, the drawings in the following description are some embodiments of the present disclosure, and for those skilled in the art, other drawings may be obtained according to these drawings without paying creative efforts.

FIG. 1 is a schematic flowchart of a content generation method provided by at least one embodiment of the present disclosure;

FIG. 2 is a schematic diagram of a startup interface of a medical examination report function provided by at least one embodiment of the present disclosure;

FIG. 3 is a schematic diagram of a conversation interface provided by at least one embodiment of the present disclosure;

FIG. 4 is a schematic diagram of an image uploading interface provided by at least one embodiment of the present disclosure;

FIG. 5 is a schematic display diagram of a report interpretation result provided by at least one embodiment of the present disclosure;

FIG. 6 is a schematic flowchart of another content generation method provided by at least one embodiment of the present disclosure;

FIG. 7 is a schematic structural diagram of a content generation apparatus provided by at least one embodiment of the present disclosure; and

FIG. 8 is a schematic structural diagram of an electronic device provided by at least one embodiment of the present disclosure.

### DETAILED DESCRIPTION

Embodiments of the present disclosure will now be described in more detail with reference to the drawings. Although certain embodiments of the present disclosure are shown in the drawings, it should be understood that the present disclosure may be implemented in various forms and should not be construed as limited to the embodiments set forth herein; on the contrary, these embodiments are provided for a more thorough and complete understanding of the present disclosure. It should be understood that the drawings and embodiments of the present disclosure are only used for exemplary purposes, and are not used to limit the protection scope of the present disclosure.

It should be understood that various steps described in method implementations of the present disclosure may be performed in different orders and/or in parallel. Furthermore, the method implementations may include additional steps and/or omit performing the illustrated steps. The scope of the present disclosure is not limited in this respect.

The term “include/comprise” and variations thereof used herein are open-ended inclusions, that is, “include/comprise but not limited to”. The term “based on” means “based at least in part on”. The term “one embodiment” means “at least one embodiment”; the term “another embodiment” means “at least one other embodiment”; the term “some embodiments” means “at least some embodiments”. Relevant definitions of other terms will be given in the following description.

It should be noted that concepts such as “first” and “second” mentioned in the present disclosure are only used to distinguish different apparatuses, modules or units, and are not used to limit the order or interdependence of functions performed by these apparatuses, modules or units.

It should be noted that the modifications of “one” and “multiple” mentioned in the present disclosure are illustrative rather than restrictive, and those skilled in the art should understand that unless clearly indicated in the context, they should be understood as “one or multiple”.

The names of messages or information exchanged between multiple apparatuses in the implementations of the present disclosure are only used for illustrative purposes, and are not used to limit the scope of these messages or information.

A medical examination report covers physical condition information obtained by various medical means, which may include test reports such as blood routine and urine routine, may also include electrocardiograms, endoscopy reports for examining internal cavities of body organs, and the like, and may further include pathological reports, function examination reports of body organs, and the like. There are mainly two types of solutions for automatically interpreting medical examination reports in the related art. One is a report interpretation solution based on a medical rule system, which mainly uses a preset medical report interpretation template to automatically generate a fixed summary based on numerical abnormalities of specific indicators (for example, exceeding a normal numerical range of the indicators). However, the medical report interpretation template used by such a method is relatively fixed, resulting in poor flexibility and readability of medical report interpretation, and it may only analyze a single indicator but lacks the ability of multi-indicator correlation analysis, resulting in low accuracy of report interpretation results. The other is a report interpretation solution based on machine learning algorithms, which uses traditional machine learning algorithms to extract and analyze important keywords or medical entities (such as disease names, drugs, symptoms, etc.) in medical examination reports, and generates brief report interpretation results, such as “elevated white blood cells”, “anemia”, and the like. However, such a method has problems such as insufficient context understanding, limited generalization ability, and poor readability due to inhumanized interpretation results.

Based on the above situation, at least one embodiment of the present disclosure provides a content generation solution to pre-train a generative model (such as a language model) with a certain scale using medical examination report samples to obtain a report interpretation model adapted to medical examination reports, and uses the report interpretation model to interpret uploaded medical examination report images. In this way, it is possible to improve the flexibility of report interpretation without setting medical rules and templates, and it is possible to use the learning ability of the report interpretation model to improve the accuracy of report interpretation results by combining relevant medical knowledge (such as non-linear relationships between multiple indicators) and user-related historical information (such as historical medical records, historical diagnosis results and symptoms, etc.), and it is also possible to use the natural language generation ability of the report interpretation model to improve the fluency and humanization of report interpretation results, thereby improving the readability of report interpretation results and user experience.

The content generation method provided by the embodiment of the present disclosure is applicable to a scenario of automatically interpreting medical examination reports, for example, a search scenario in the medical field, and may also be applicable to a human-computer dialogue scenario in the medical field. The method may be performed by a content generation apparatus, which may be implemented by software and/or hardware, and the apparatus may be integrated in an electronic device having a client (such as an application, a mini-program, or a web page, etc.) with a function of implementing the above scenarios. The electronic device may include, but is not limited to, a smartphone, a personal digital assistant (PDA), a tablet personal computer (Tablet PC), a laptop computer, a desktop computer, or a medical self-service terminal device, and the like.

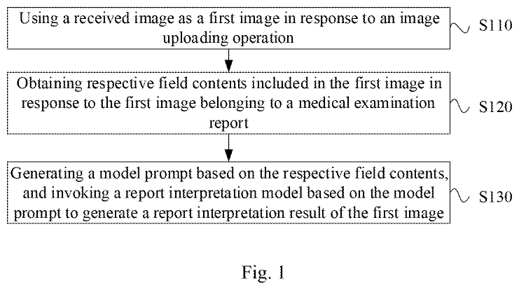

FIG. 1 shows a schematic flowchart of a content generation method provided by an embodiment of the present disclosure. As shown in FIG. 1, the content generation method may include the following steps.

S110, using a received image as a first image in response to an image uploading operation.

The image uploading operation is an interaction operation used to upload an image, which may be implemented through a preset interaction control with an image uploading function. The first image is an image to be interpreted and subjected to content generation and other processing.

Specifically, the electronic device may provide the user with various forms of interaction entrances, such as an interaction control or an interaction prompt for uploading images. The user may perform the image uploading operation through these interaction entrances. The electronic device may use a received image as the first image for this processing in response to the image uploading operation.

In some embodiments, S110 includes: displaying an image uploading interface in response to a triggering operation on a report interpretation control, and displaying an image uploading control in the image uploading interface; and using a received image as the first image in response to a triggering operation on the image uploading control.

The report interpretation control is an interaction control corresponding to an interaction entrance used to trigger a report interpretation function, for example, an interaction button with the above function. The image uploading interface is a visual interface that displays relevant prompt information and interaction controls for uploading images, which may be a page, a floating window or a floating layer, etc. The image uploading control is an interaction control with an image uploading function, for example, a “take a photo” button or an interaction button of “select from an album”, etc.

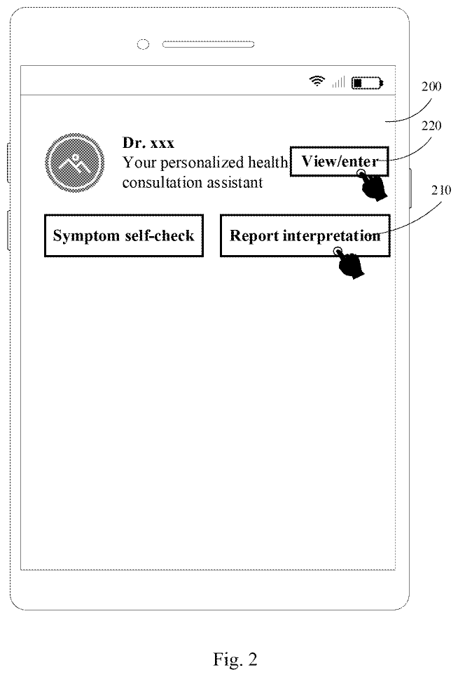

Specifically, referring to FIG. 2, the client of medical examination report interpretation may be implemented in any form such as a mini-program, an application, or a web page. When the user starts the above client, the electronic device may display a startup interface 200 of the client, and display brief information of the client (such as an icon and an introduction of “Dr. xxx: your personalized health consultation assistant”), a report interpretation control 210, and a conversation entry control 220 (such as a “view/enter” control) for entering a conversation page of the client in the startup interface 200. The user may perform a triggering operation on the report interpretation control 210 to trigger the interpretation function of the medical examination report, so that the electronic device displays the image uploading interface.

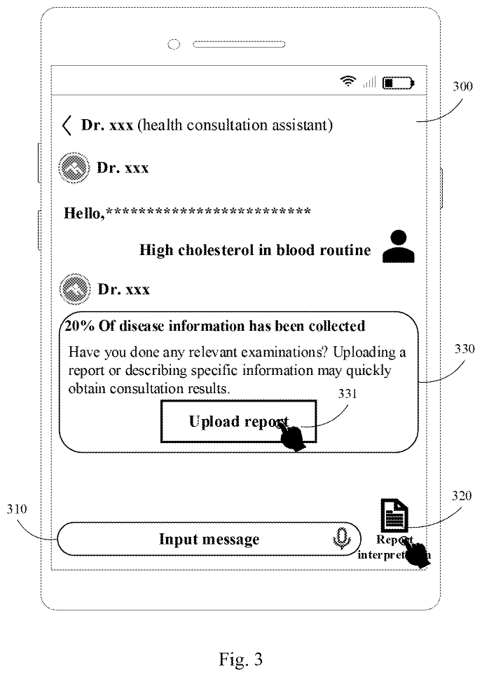

In addition, the user may also perform a triggering operation on the above conversation entry control 220 to enter a conversation interface 300 for human-computer conversation as shown in FIG. 3. The conversation interface 300 is used to display instant conversation content between the user and the client. The report interpretation control 320 may be displayed in a peripheral area of an input box 310 for conversation content in the conversation interface 300, or a top area of the conversation interface 300, etc. After the user performs the triggering operation on the report interpretation control 320, the electronic device may display the image uploading interface.

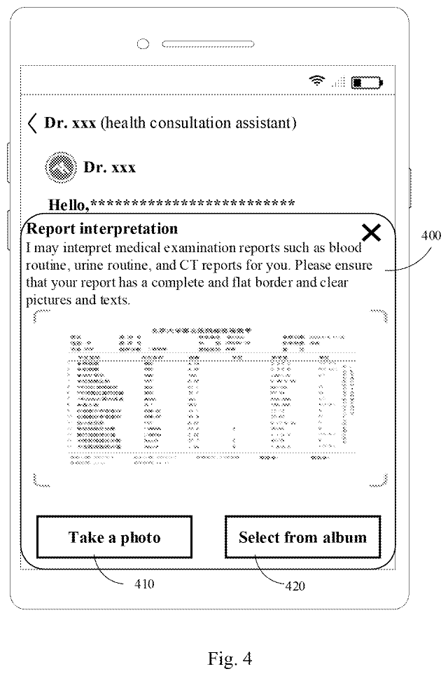

The above image uploading interface may be as shown in a floating window 400 in FIG. 4, in which prompt information (such as function introduction, interpretable medical examination report types, requirements for uploaded images, etc.) of the report interpretation function, an example of an uploaded image, and an image uploading control may be displayed. The image uploading control may be a “take a photo” button 410 that triggers an image capturing function, and/or a “select from an album” button 420 that triggers an image selection function, and the like.

After the user performs the triggering operation on the “take a photo” button 410 or the “select from an album” button 420, the electronic device may execute a corresponding image capturing process or image selection process to obtain the first image used by the user. In this way, the first image may be obtained more quickly through the interaction entrance of the explicit interaction control.

S120, obtaining respective field contents included in the first image in response to the first image belonging to a medical examination report.

Specifically, the first image uploaded by the user may not be a medical examination report, and at this time, the electronic device cannot correctly execute the report interpretation function. Therefore, the electronic device first uses a machine learning model or a matching rule combined with medical report characteristics to identify whether the first image belongs to a medical examination report. If the first image is not a medical examination report, this process ends, and the user is prompted with a relevant error. If the first image is a medical examination report, information extraction may be performed on the first image to extract fields with report analysis value and field values thereof from the first image as the respective field contents. For example, for a blood routine test report, field contents such as age, gender, specimen type, test item name, test result of the test item, reference value range of the test item, and unit of the test result may be extracted from the first image.

S130, generating a model prompt based on the respective field contents, and invoking a report interpretation model based on the model prompt to generate a report interpretation result of the first image.

The model prompt (prompt) is a piece of text content input by the user to the model, which is used to guide the model to generate a specific output. The report interpretation model is obtained by fine-tuning a pre-trained generative model using medical examination report samples in advance. The generative model here may be a language model with a certain scale, which may be a general base model pre-trained with large-scale diverse training samples, or a medical base model pre-trained specifically with medical-related training samples. The medical examination report samples may be field contents corresponding to various types of medical examination reports and corresponding expert interpretation results.

Specifically, in order to improve the adaptability of the generative model to the interpretation scenario of medical examination reports, in the embodiments of the present disclosure, a certain amount of medical examination report samples may be obtained in advance, and the generative model may be fine-tuned with these medical examination report samples to obtain a report interpretation model that is more suitable for the interpretation scenario of medical examination reports. In this way, the generative model may have larger-scale training, and the generalization ability of the model may be improved. In addition, the model may further learn the non-linear relationship between multiple medical examination indicators on the basis of various knowledge it has learned, so as to have the ability to comprehensively analyze the relationship between the multiple medical examination indicators and corresponding diseases, thereby improving the accuracy of medical report interpretation results. Moreover, the natural language processing ability of the generative model may be used to improve the fluency and humanization of the output medical report interpretation results, thereby improving the readability of the report interpretation results.

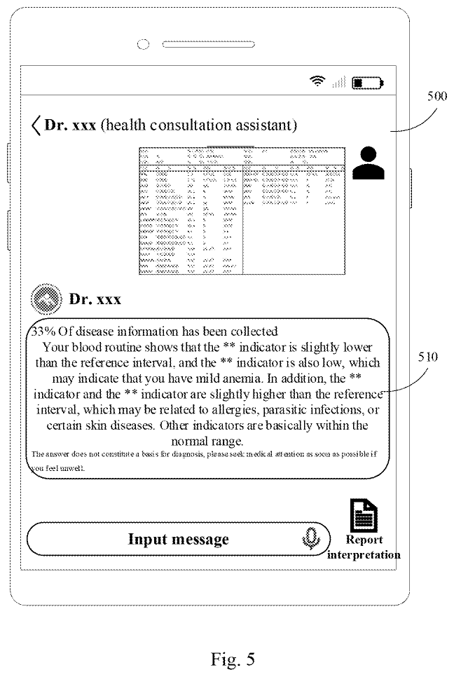

After obtaining the respective field contents in the first image, the electronic device may use these field contents to fill in placeholders in a pre-built prompt template to form the model prompt. Then, the electronic device inputs the model prompt into the report interpretation model to trigger the operation of the model, and outputs an interpretation summary of the model on the first image, that is, the report interpretation result, according to an output format in the model prompt. After that, the electronic device may display the report interpretation result in the conversation interface. As shown in FIG. 5, after the user uploads the first image, the electronic device may display a report interpretation result 510 in a conversation interface 500.

According to the content generation method provided by the above embodiments of the present disclosure, a received image is used as a first image in response to an image uploading operation; respective field contents included in the first image are obtained in response to the first image belonging to a medical examination report; a model prompt is generated based on the respective field contents, and a report interpretation model, which is obtained by fine-tuning a pre-trained generative model using medical examination report samples in advance, is invoked based on the model prompt to generate a report interpretation result of the first image. In this way, the image of the medical examination report is interpreted by using the report interpretation model adapted to the medical field. On the one hand, the generalization ability of the report interpretation model is improved by using a widely-trained generative model with a certain scale, and the flexibility of medical report interpretation and the adaptability to various medical examination reports are improved without predefined rules or templates. On the other hand, the learning ability and context analysis ability of the report interpretation model are used to automatically capture complex non-linear relationships between multiple medical indicators, and generate more comprehensive, smooth and user-friendly medical report interpretation results by combining medical history, symptoms and examination data, thereby improving the readability and accuracy of the interpretation results, and improving the user experience.

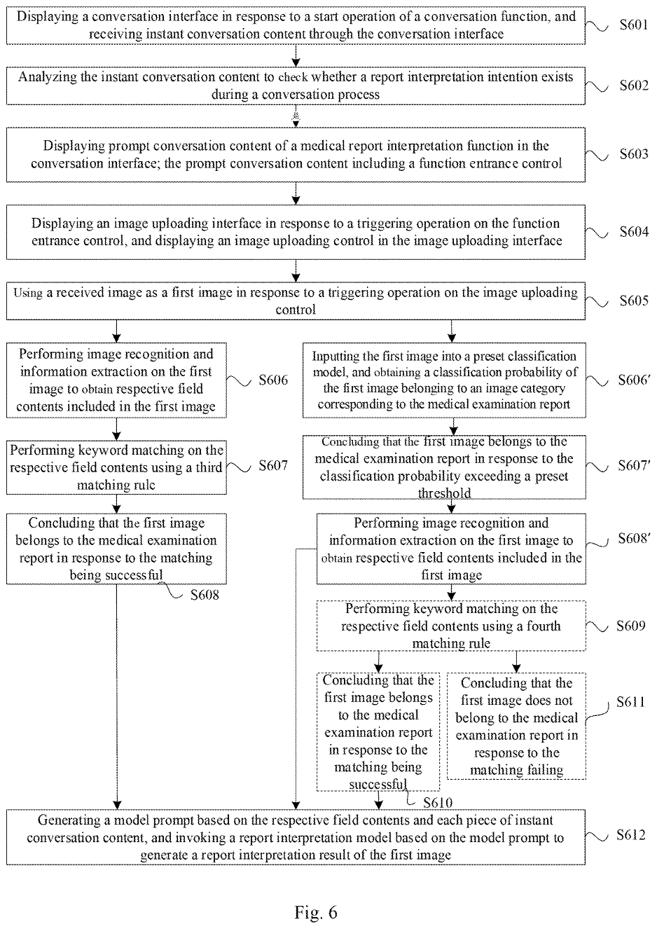

FIG. 6 is a schematic flowchart of another content generation method provided by at least one embodiment of the present disclosure. In some embodiments, the content generation method may further specify “using a received image as a first image in response to an image uploading operation” . On this basis, “generating a model prompt based on the respective field contents” may be further specified. In some other embodiments, the content generation method may further specify “obtaining respective field contents included in the first image in response to the first image belonging to a medical examination report”. Referring to FIG. 6, the content generation method specifically includes the following steps.

S601, displaying a conversation interface in response to a start operation of a conversation function, and receiving instant conversation content through the conversation interface.

The instant conversation content is the specific content of conversation messages obtained/feedback in real time in a human-computer conversation.

Specifically, still referring to FIG. 2, when the user performs a triggering operation on the conversation entry control 220, it indicates that the user wants to start the human-computer conversation function for health consultation, and the electronic device may display the conversation interface 300 as shown in FIG. 3 in response to the triggering operation. The user may input a medical question that needs to be consulted through the conversation interface 300, and the client may feed back the medical question through a corresponding language model in the medical field at the backend. These medical questions and feedbacks may be used as the instant conversation content.

S602, analyzing the instant conversation content to check whether a report interpretation intention exists during a conversation process.

Specifically, in addition to the explicit interaction controls mentioned in the foregoing embodiments, the interaction entrance of the medical examination report interpretation function may automatically check whether the user has a need/intention to interpret a medical examination report (i.e., the report interpretation intention) based on the conversation content of human-computer conversation. In this way, the needs of the user may be better identified, so that the user may be provided with the interaction entrance of the image uploading function in a timely manner, so as to better guide the user to use the medical examination report interpretation function, thereby further improving the user experience. Based on this, the electronic device may perform intention recognition on each piece of instant conversation content obtained in real time to check whether the report interpretation intention exists during the conversation process.

In some embodiments, S602 may be implemented as: analyzing the instant conversation content using a fifth matching rule and/or a conversation analysis model to check whether the report interpretation intention exists during the conversation process.

The fifth matching rule is a preset keyword matching rule, which may be set according to relevant keywords of the medical examination report and a matching business scenario. For example, if the matching business scenario is a rough matching scenario, the fifth matching rule may be implemented as a keyword matching rule (i.e., a first matching rule) related to the medical examination report with relatively weak matching constraints, such as a rule for matching specific keywords such as “medical report”, “physical examination”, “test”, “blood test data”, “blood routine”, and “urine routine” and similar keywords thereof. For another example, if the matching business scenario is an exact matching scenario, the fifth matching rule may be implemented as a keyword matching rule (i.e., a second matching rule) related to the medical examination report with relatively strong matching constraints, such as a rule for matching specific keywords such as “medical report”, “physical examination”, “test”, “blood test data”, “blood routine”, and “urine routine”. The conversation analysis model is a pre-trained language model, and its function is to identify intention/requirement of a medical examination report through text analysis.

Specifically, the electronic device may use the fifth matching rule, the conversation analysis model, a combination of the fifth matching rule and the conversation analysis model, etc. to analyze each piece of instant conversation content to identify whether the user has a report interpretation intention during the conversation process.

In some examples, the analyzing the instant conversation content using the fifth matching rule to check whether there is the report interpretation intention during the conversation process may be implemented as follows: the electronic device may perform keyword matching on each piece of instant conversation content using the predefined fifth matching rule. In response to the matching being successful, there is the report interpretation intention during the conversation process; in response to the matching failing, there is no report interpretation intention during the conversation process. In this way, a relatively simple keyword matching rule may be used to obtain the intention, which reduces the implementation cost of report interpretation intention recognition to a certain extent and improves the efficiency of report interpretation intention recognition.

In some other examples, the analyzing the instant conversation content using the conversation analysis model to check whether the report interpretation intention exists during the conversation process may be implemented as follows: the electronic device may use each piece of instant conversation content to generate a model prompt, and input the model prompt into the conversation analysis model to trigger the operation of the model, and finally output a model output result of whether the report interpretation intention exists, for example, whether a medical report is mentioned and whether the report interpretation is required, etc. If the model output result is that the medical report is not mentioned, or the medical report is mentioned but the report interpretation is not required, there is no report interpretation intention during the conversation process; on the contrary, if the model output result is that the medical report is mentioned and the report interpretation is required, the report interpretation intention exists during the conversation process. In this way, the accuracy of intention recognition may be improved to a certain extent.

In some other examples, the analyzing the instant conversation content using the fifth matching rule and the conversation analysis model to check whether the report interpretation intention exists during the conversation process may be implemented as follows: performing keyword matching on each piece of instant conversation content using the first matching rule; and in response to the matching being successful, invoking the conversation analysis model based on first content to generate a model output result.

The model output result represents whether the report interpretation intention exists during the conversation process. The first content includes at least one selected from the group consisting of each piece of instant conversation content, a keyword that is successfully matched, and the instant conversation content corresponding to the keyword.

Specifically, in order to further improve the accuracy of intention recognition on the basis of controlling implementation costs and efficiency, the electronic device may first perform keyword matching on each piece of instant conversation content using the first matching rule with relatively low matching accuracy. If the matching fails, it indicates that the probability of each piece of instant conversation content having the report interpretation intention is very small, and there is no report interpretation intention, and this report interpretation process may end. In this way, there is no need to execute the conversation analysis model, which saves running costs. If the matching is successful, it indicates that the probability of each piece of instant conversation content having the report interpretation intention is relatively high. Then, a model prompt may be constructed using the first content, and the model prompt may be input into the conversation analysis model for intention recognition to obtain the model output result. If the model output result is that the medical report is mentioned and the report interpretation is required, there is the report interpretation intention during the conversation process. If the model output result is that the medical report is not mentioned, or the medical report is mentioned but the report interpretation is not required, there is no report interpretation intention during the conversation process. In this way, the accuracy of intention recognition may be further improved.

In some other examples, the analyzing the instant conversation content using the fifth matching rule and the conversation analysis model to check whether the report interpretation intention exists during the conversation process may be implemented as follows: invoking the conversation analysis model based on each piece of instant conversation content to generate a model output result; and in response to the model output result representing that the report interpretation intention exists, performing keyword matching on each piece of instant conversation content using the second matching rule; in response to the matching being successful, concluding that the report interpretation intention exists during the conversation process; and in response to the matching failing, concluding that there is no report interpretation intention during the conversation process.

Specifically, considering that the accuracy of intention recognition by the conversation analysis model is higher than that of the first matching rule, but there may also be certain recognition errors for various reasons such as insufficient training and model hallucination. In this embodiment, a model prompt may be constructed using each piece of instant conversation content, and the conversation analysis model may be invoked using the model prompt to generate a model output result. If the model output result represents that there is no report interpretation intention, this report interpretation process ends. If the model output result represents that the report interpretation intention exists, it indicates that there is a great need for report interpretation during the conversation process. At this time, the second matching rule may be further used to perform stricter keyword matching on each piece of instant conversation content. If the matching fails, it indicates that the model recognition is incorrect, and there is no report interpretation intention during the conversation process. If the matching is successful, the report interpretation intention exists during the conversation process. In this way, the recognition accuracy of the report interpretation intention may be further improved, thereby further improving the timeliness of subsequently displaying the image uploading entrance and avoiding interference to the user, and further improving the user experience.

S603, in response to the report interpretation intention existing during a conversation process, displaying prompt conversation content of a medical report interpretation function in the conversation interface, the prompt conversation content including a function entry control.

The prompt conversation content is conversation content used to prompt the user to upload an image of the medical examination report, which may include a relevant prompt text and a function entry control. The function entry control is an interaction control corresponding to an entrance to enter the medical report interpretation function, for example, an “upload report” button.

Specifically, if there is no report interpretation intention during the conversation process, the electronic device may continue to answer and feedback the medical question input by the user. If the report interpretation intention exists during the conversation process, the electronic device may display the prompt conversation content in the conversation interface as a feedback to the instant conversation content of the user.

Still referring to FIG. 3, the electronic device holds that there is a report interpretation requirement after performing intention recognition on the instant conversation content of “high cholesterol in blood routine”, then the prompt conversation content 330 may be displayed after the instant conversation content in the conversation interface 300, and an “upload report” button 331 may be displayed in the prompt conversation content 330 to guide the user to upload the image of the medical examination report.

S604, displaying an image uploading interface in response to a triggering operation on the function entry control, and displaying an image uploading control in the image uploading interface.

Specifically, if the user performs the triggering operation on the function entry control exemplified by the “upload report” button 331, the electronic device may display the image uploading interface as shown in FIG. 4 in response to the triggering operation, and display the image uploading control in the image uploading interface.

S605, using a received image as a first image in response to a triggering operation on the image uploading control.

S606, performing image recognition and information extraction on the first image to obtain respective field contents included in the first image.

Specifically, after obtaining the first image, the electronic device may use image recognition and information extraction algorithms such as optical character recognition to recognize and extract fields included in the first image to obtain the respective field contents.

It should be noted that in the process of image recognition and information extraction, user personal information involved may be ignored to protect user privacy.

S607, performing keyword matching on the respective field contents using a third matching rule.

The third matching rule is another preset keyword matching rule with relatively weak matching constraints, which may be set according to the content characteristics of the medical examination report, for example, by keywords unique to the medical examination report such as “hospital”, “indicator unit”, “physician/doctor”, “indicator value”, “reference range/reference value”, etc. to set a keyword matching rule (such as whether the keywords exist, the number of times the keywords are successfully matched, etc.).

Specifically, the electronic device may first perform keyword matching on the respective field contents using the third matching rule to check whether the first image has specific content of the medical examination report, thereby checking whether it belongs to the medical examination report.

S608, concluding that the first image belongs to the medical examination report in response to the matching being successful.

Specifically, if there is a keyword that is successfully matched, it indicates that the first image belongs to the medical examination report, and the subsequent steps are continued. If there is no keyword that is successfully matched, that is, the matching fails, it indicates that the first image does not belong to the medical examination report, and this process ends, thereby reducing resource waste and improving running efficiency.

S606′, inputting the first image into a preset classification model, and obtaining a classification probability of the first image belonging to an image category corresponding to the medical examination report.

The preset classification model is a pre-trained model with a classification function, which may predict the probability of the first image belonging to the medical examination report.

Specifically, after obtaining the first image, the first image may also be first input into the preset classification model, and the classification probability of the first image belonging to the image category corresponding to the medical examination report is output through the operation of the model.

S607′, concluding that the first image belongs to the medical examination report in response to the classification probability exceeding a preset threshold.

The preset threshold is a preset probability critical value.

Specifically, if the classification probability does not exceed the preset threshold, it indicates that the first image does not belong to the medical examination report, and this process ends. On the contrary, if the classification probability exceeds the preset threshold, the first image belongs to the medical examination report.

S608′, performing image recognition and information extraction on the first image to obtain respective field contents included in the first image.

Specifically, the electronic device may use image recognition and information extraction algorithms such as optical character recognition to recognize and extract fields included in the first image to obtain the respective field contents.

S609, performing keyword matching on the respective field contents using a fourth matching rule.

The fourth matching rule is another preset keyword matching rule with relatively stronger matching constraints than the third matching rule, which may be set according to the content characteristics of the medical examination report.

Specifically, on the basis of preliminarily concluding that the first image belongs to the medical examination report by the preset classification model, keyword matching processing may be further performed on the respective field contents in the first image using the fourth matching rule to further check whether the first image belongs to the medical examination report.

S610, concluding that the first image belongs to the medical examination report in response to the matching being successful.

S611, concluding that the first image does not belong to the medical examination report in response to the matching failing.

S612, generating a model prompt based on the respective field contents and each piece of instant conversation content, and invoking a report interpretation model based on the model prompt to generate a report interpretation result of the first image.

Specifically, on the basis of concluding that the user has a report interpretation requirement based on each piece of instant conversation content, the model prompt may be constructed jointly using the respective field contents in the first image and each piece of instant conversation content, and the report interpretation model may be invoked using the model prompt. In this way, conversation context content (which may include various information such as the user's medical history, symptoms, and historical medical records) during this conversation process may be further provided for the report interpretation model as supplementary information, thereby increasing the amount of information that may be captured by the model, and further improving the accuracy of report interpretation.

According to the content generation method provided by the above embodiments, a conversation interface is displayed in response to a start operation of a conversation function, and instant conversation content is received through the conversation interface; the instant conversation content is analyzed to check whether there is a report interpretation intention during a conversation process; in response to the report interpretation intention existing during a conversation process, prompt conversation content of a medical report interpretation function is displayed in the conversation interface, the prompt conversation content including a function entry control; an image uploading interface is displayed in response to a triggering operation on the function entry control, and an image uploading control is displayed in the image uploading interface; and a received image is used as a first image in response to a triggering operation on the image uploading control. In this way, the needs of the user may be better identified, and the user may be provided with an interaction entrance of an image uploading function in a timely manner, so as to better guide the user to use the medical examination report interpretation function, thereby further improving the user experience. Moreover, by generating a model prompt based on the respective field contents and each piece of instant conversation content, conversation context content during this conversation process may be further provided for the report interpretation model as supplementary information, thereby increasing the amount of information that may be captured by the model, and further improving the accuracy of report interpretation. In addition, through the combination of image recognition, information extraction and the third matching rule, resource waste may be reduced to a certain extent and image obtaining efficiency may be improved on the basis of recognizing whether the first image is a medical examination report. Through the combination of the preset classification model, and image recognition and information extraction, the accuracy of image obtaining may be improved to a certain extent on the basis of recognizing whether the first image is a medical examination report. Through the combination of the preset classification model, the image recognition and information extraction and the fourth matching rule, the accuracy of image obtaining may be further improved on the basis of recognizing whether the first image is a medical examination report, thereby further reducing resource waste of report interpretation and improving running efficiency.

The following is an embodiment of a content generation apparatus provided by an embodiment of the present invention. The apparatus belongs to the same inventive concept as the content generation method of the above embodiments. For details not described in detail in the embodiment of the content generation apparatus, reference may be made to the embodiment of the above content generation method.

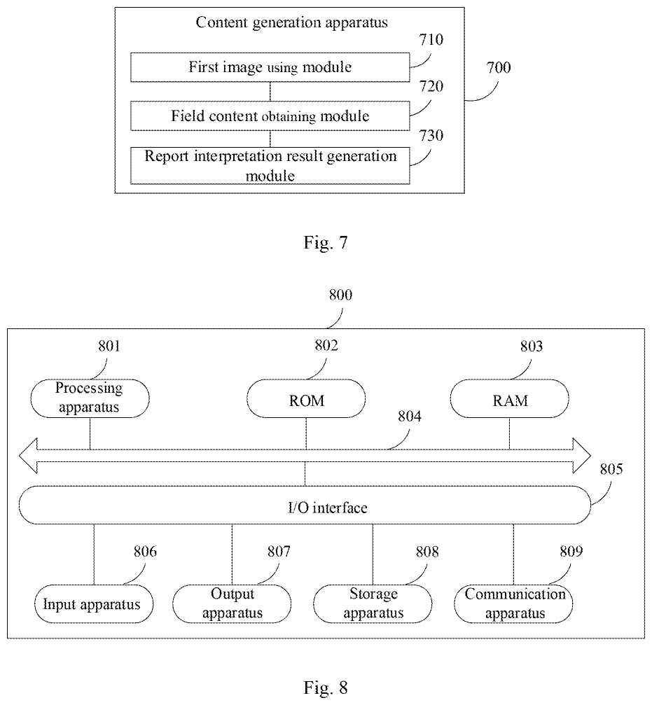

FIG. 7 is a schematic structural diagram of a content generation apparatus provided by an embodiment of the present disclosure. As shown in FIG. 7, the content generation apparatus 700 may include:
a first image using module 710, configured to use a received image as a first image in response to an image uploading operation;
a field content obtaining module 720, configured to obtain respective field contents included in the first image in response to the first image belonging to a medical examination report; and
a report interpretation result generation module 730, configured to generate a model prompt based on the respective field contents, and invoke a report interpretation model based on the model prompt to generate a report interpretation result of the first image; where the report interpretation model is obtained by fine-tuning a pre-trained generative model using medical examination report samples in advance.

According to the content generation apparatus provided by the above embodiments of the present disclosure, a received image is used as a first image in response to an image uploading operation; respective field contents included in the first image are obtained in response to the first image belonging to a medical examination report; a model prompt is generated based on the respective field contents, and a report interpretation model, which is obtained by fine-tuning a pre-trained generative model using medical examination report samples in advance, is invoked based on the model prompt to generate a report interpretation result of the first image. In this way, the image of the medical examination report is interpreted by using the report interpretation model adapted to the medical field. On the one hand, the generalization ability of the report interpretation model is improved by using a widely-trained generative model with a certain scale, and the flexibility of medical report interpretation and the adaptability to various medical examination reports are improved without predefined rules or templates. On the other hand, the learning ability and context analysis ability of the report interpretation model are used to automatically capture complex non-linear relationships between multiple medical indicators, and generate more comprehensive, smooth and user-friendly medical report interpretation results by combining medical history, symptoms and examination data, thereby improving the readability and accuracy of the interpretation results, and improving the user experience.

In some embodiments, the first image using module 710 includes:
an instant conversation content reception sub-module, configured to display a conversation interface in response to a start operation of a conversation function, and receive instant conversation content through the conversation interface;
a report interpretation intention obtaining sub-module, configured to analyze the instant conversation content to determine whether there is a report interpretation intention during a conversation process;
a prompt conversation content display sub-module, configured to, in response to the report interpretation intention existing during a conversation process, display prompt conversation content of a medical report interpretation function in the conversation interface, the prompt conversation content including a function entry control;
an image uploading control display sub-module, configured to display an image uploading interface in response to a triggering operation on the function entry control, and display an image uploading control in the image uploading interface; and
a first image using sub-module, configured to use a received image as the first image in response to a triggering operation on the image uploading control.

In some embodiments, the report interpretation intention obtaining sub-module is further configured to:
analyze the instant conversation content using a fifth matching rule and/or a conversation analysis model to check whether the report interpretation intention exists during the conversation process, the conversation analysis model being a pre-trained language model.

Further, the report interpretation intention obtaining sub-module is further configured to:
perform keyword matching on each piece of instant conversation content using the first matching rule; and
in response to the matching being successful, invoke the conversation analysis model based on first content to generate a model output result, the model output result representing whether the report interpretation intention exists during the conversation process, the first content including at least one selected from the group consisting of each piece of instant conversation content, a keyword that is successfully matched, and instant conversation content corresponding to the keyword.

Optionally, the report interpretation intention obtaining sub-module is further configured to:
invoke the conversation analysis model based on each piece of instant conversation content to generate a model output result;
in response to the model output result representing that there is the report interpretation intention, perform keyword matching on each piece of instant conversation content using the second matching rule;
in response to the matching being successful, there is the report interpretation intention during the conversation process; and
in response to the matching failing, there is no report interpretation intention during the conversation process.

In some embodiments, the report interpretation result generation module 730 is further configured to:
generate the model prompt based on the respective field contents and each piece of instant conversation content.

In some embodiments, the field content obtaining module 720 is further configured to:
perform image recognition and information extraction on the first image to obtain the respective field contents included in the first image;
perform keyword matching on the respective field contents using the third matching rule; and
obtain that the first image belongs to the medical examination report in response to the matching being successful.

In some other embodiments, the field content obtaining module 720 is further configured to:
input the first image into a preset classification model, and obtain a classification probability of the first image belonging to an image category corresponding to the medical examination report;
obtain that the first image belongs to the medical examination report in response to the classification probability exceeding a preset threshold; and
perform image recognition and information extraction on the first image to obtain the respective field contents included in the first image.

Further, in some embodiments, the field content obtaining module 720 is further configured to:
after performing image recognition and information extraction on the first image to obtain the respective field contents included in the first image, perform keyword matching on the respective field contents using the fourth matching rule;
obtain that the first image belongs to the medical examination report in response to the matching being successful; and
obtain that the first image does not belong to the medical examination report in response to the matching failing.

In some other embodiments, the first image using module 710 is further configured to:
display an image uploading interface in response to a triggering operation on a report interpretation control, and display an image uploading control in the image uploading interface; and
using a received image as the first image in response to a triggering operation on the image uploading control.

The content generation apparatus provided by the embodiment of the present invention may perform the content generation method provided by any embodiment of the present invention, and has corresponding functional modules and beneficial effects for performing the method.

It is worth noting that in the embodiment of the above content generation apparatus, the included units and modules are only divided according to functional logic, but are not limited to the above division, as long as the corresponding functions may be realized; in addition, the specific names of the functional units are only for the convenience of distinguishing from each other, and are not used to limit the protection scope of the present disclosure.

At least one embodiment of the present disclosure further provides an electronic device, which may include a processor and a memory, the memory may be used to store executable instructions. The processor may be configured to read the executable instructions from the memory, and execute the executable instructions to implement the content generation method in the above embodiments.

FIG. 8 is a schematic structural diagram of an electronic device provided by an embodiment of the present disclosure.

As shown in FIG. 8, the electronic device 800 may include a processing apparatus 801 (such as a central processing unit, a graphics processing unit, etc.), which may perform various appropriate actions and processing according to a program stored in a read-only memory (ROM) 802 or a program loaded into a random access memory (RAM) 803 from a storage apparatus 808. The RAM 803 further stores various programs and data required for operations of the electronic device 800. The processing apparatus 801, the ROM 802, and the RAM 803 are connected to each other through a bus 804. An input/output interface (I/O interface) 805 is also connected to the bus 804.

Usually, the following apparatuses may be connected to the I/O interface 805: an input apparatus 806 including, for example, a touch screen, a touchpad, a keyboard, a mouse, a camera, a microphone, an accelerometer, or a gyroscope; an output apparatus 807 including, for example, a liquid crystal display (LCD), a speaker, a vibrator, etc.; the storage apparatus 808 including, for example, a magnetic tape, a hard disk, etc.; and a communication apparatus 809. The communication apparatus 809 may allow the electronic device 800 to perform wireless or wired communication with other devices to exchange data.

It should be noted that the electronic device 800 shown in FIG. 8 is only an example, and should not impose any limitation on the function and scope of use of the embodiments of the present disclosure. That is, although FIG. 8 shows the electronic device 800 with various apparatuses, it should be understood that it is not required to implement or have all the shown apparatuses. Alternatively, more or fewer apparatuses may be implemented or provided.

In particular, according to the embodiment of the present disclosure, the process described above with reference to the flowchart may be implemented as a computer software program. For example, the embodiment of the present disclosure includes a computer program product, which includes a computer program carried on a non-transitory computer-readable medium, and the computer program includes program code for performing the method shown in the flowchart. In such an embodiment, the computer program may be downloaded and installed from a network through the communication apparatus 809, or installed from the storage apparatus 808, or installed from the ROM 802. When the computer program is executed by the processing apparatus 801, the above-mentioned functions defined in the content generation method according to any one of the embodiments of the present disclosure are executed.

At least one embodiment of the present disclosure further provides a non-transitory computer-readable storage medium, the non-transitory computer-readable storage medium stores a computer program, the computer program, when executed by a processor, causes the processor to implement the content generation method according to any one of the embodiments of the present disclosure.

It should be noted that the above non-transitory computer-readable medium in the present disclosure may be a computer-readable signal medium, a computer-readable storage medium, or any combination of the two. The non-transitory computer-readable storage medium may be, for example, a system, apparatus or device including, but not limited to, an electronic, magnetic, optical, electromagnetic, infrared, or semiconductor one, or any combination of the above. More specific examples of the computer-readable storage medium may include, but are not limited to: an electrically connected portable computer magnetic disk with one or more wires, a hard disk, a random access memory (RAM), a read-only memory (ROM), an erasable programmable read-only memory (EPROM), a flash memory, an optical fiber, a portable compact disc read-only memory (CD-ROM), an optical storage device, a magnetic storage device, or any suitable combination of the above. In the present disclosure, a computer-readable storage medium may be any tangible medium that contains or stores a program, which may be used by or in combination with an instruction execution system, apparatus, or device. In the present disclosure, a computer-readable signal medium may include a data signal propagated in a baseband or as a part of a carrier wave, and computer-readable program code is carried therein. This propagated data signal may take multiple forms, including but not limited to an electromagnetic signal, an optical signal, or any suitable combination of the above. The computer-readable signal medium may also be any computer-readable medium other than the computer-readable storage medium, and the computer-readable signal medium may send, propagate, or transmit a program used by or in combination with an instruction execution system, apparatus, or device. The program code contained on the computer-readable medium may be transmitted by any suitable medium, including but not limited to: a wire, an optical cable, a radio frequency (RF), etc., or any suitable combination of the above.

In some implementations, the client and the server may communicate using any currently known or future developed network protocol, such as the hypertext transfer protocol (HTTP), and may be interconnected with any form or medium of digital data communication (for example, a communication network). Examples of the communication network include a local area network (LAN), a wide area network (WAN), an internet (for example, the Internet), a peer-to-peer network (for example, an Ad-Hoc network), and any network currently known or to be developed in the future.

The above computer-readable medium may be included in the above electronic device, or may exist alone without being assembled into the electronic device.

The above computer-readable medium carries one or more programs, which, when executed by the electronic device, causes the electronic device to perform the content generation method according to any one of the embodiments of the present disclosure.

In the embodiments of the present disclosure, computer program code for performing operations of the present disclosure may be written in one or more programming languages or a combination thereof, the above programming languages including but not limited to object-oriented programming languages such as Java, Smalltalk, C++, and conventional procedural programming languages such as “C” language or similar programming languages. The program code may be executed entirely on a user's computer, partly executed on a user's computer, executed as an independent software package, partly executed on a user's computer and partly executed on a remote computer, or entirely executed on a remote computer or server. In the case of involving a remote computer, the remote computer may be connected to a user's computer through any kind of network, including a local area network (LAN) or a wide area network (WAN), or it may be connected to an external computer (for example, connected through the Internet using an Internet service provider).

The flowchart and block diagrams in the drawings illustrate the possibly implemented architectures, functions, and operations of the device, method and computer program product according to various embodiments of the present disclosure. In this regard, each block in the flowchart or block diagrams may represent a module, program segment, or part of code, which includes one or more executable instructions for implementing the specified logical functions. It should also be noted that, in some alternative implementations, the functions marked in the blocks may also occur in an order different from that marked in the drawings. For example, two blocks shown in succession may actually be executed substantially in parallel, or they may sometimes be executed in the reverse order, depending on the functions involved. It should also be noted that each block in the block diagrams and/or flowcharts, and a combination of the blocks in the block diagrams and/or flowcharts may be implemented by a dedicated hardware-based system that executes specified functions or operations, or may be implemented by a combination of dedicated hardware and computer instructions.

The functions described above may be performed at least partially by one or more hardware logic components. For example, without limitation, exemplary types of hardware logic components that may be used include: a field programmable gate array (FPGA), an application specific integrated circuit (ASIC), an application specific standard parts (ASSP), a system on chip (SOC), a complex programmable logic device (CPLD), etc.

The above description is only preferred embodiments of the present disclosure and an explanation of the applied technical principles. Those skilled in the art should understand that the disclosed scope involved in the present disclosure is not limited to the technical solutions formed by the specific combination of the above technical features, and should also cover other technical solutions formed by arbitrary combination of the above technical features or their equivalent features without departing from the above disclosed concept. For example, a technical solution formed by replacing the above features with technical features with similar functions disclosed in the present disclosure (but not limited to).

Furthermore, although the operations have been described in a particular order, this should not be understood as requiring the operations to be performed in the particular order shown or in a sequential order. Under certain circumstances, multitasking and parallel processing may be advantageous. Likewise, although the above discussion contains a number of specific implementation details, these should not be construed as limiting the scope of the present disclosure. Certain features that are described in the context of separate embodiments may also be implemented in combination in a single embodiment. Conversely, various features described in the context of a single embodiment may also be implemented in multiple embodiments individually or in any suitable sub-combinations.

Although the subject matter has been described in language specific to structural features and/or logical actions of the method, it should be understood that the subject matter defined in the appended claims is not necessarily limited to the specific features or actions described above. Conversely, the specific features and actions described above are merely exemplary forms for implementing the claims.

1. A content generation method, comprising:
using a received image as a first image in response to an image uploading operation;
obtaining respective field contents comprised in the first image in response to the first image belonging to a medical examination report; and
generating a model prompt based on the respective field contents, and invoking a report interpretation model based on the model prompt to generate a report interpretation result of the first image; wherein the report interpretation model is obtained by fine-tuning a pre-trained generative model using medical examination report samples in advance.

2. The method of claim 1, wherein the using a received image as a first image in response to an image uploading operation comprises:
displaying a conversation interface in response to a start operation of a conversation function, and receiving instant conversation content through the conversation interface;
analyzing the instant conversation content to check whether a report interpretation intention exists during a conversation process;
in response to the report interpretation intention existing during a conversation process, displaying prompt conversation content of a medical report interpretation function in the conversation interface, the prompt conversation content comprising a function entry control;
displaying an image uploading interface in response to a triggering operation on the function entry control, and displaying an image uploading control in the image uploading interface; and
using the received image as the first image in response to a triggering operation on the image uploading control.

3. The method of claim 2, wherein the analyzing the instant conversation content to check whether a report interpretation intention exists during a conversation process comprises:
analyzing the instant conversation content using a fifth matching rule and/or a conversation analysis model to check whether the report interpretation intention exists during the conversation process, the conversation analysis model being a pre-trained language model.

4. The method of claim 3, wherein the analyzing the instant conversation content using a fifth matching rule and a conversation analysis model to check whether the report interpretation intention exists during the conversation process comprises:
performing keyword matching on each piece of instant conversation content using a first matching rule; and
in response to the matching being successful, invoking the conversation analysis model based on first content to generate a model output result, the model output result representing whether the report interpretation intention exists during the conversation process, the first content comprising at least one selected from the group consisting of each piece of instant conversation content, a keyword that is successfully matched, and the instant conversation content corresponding to the keyword.

5. The method of claim 3, wherein the analyzing the instant conversation content using a fifth matching rule and a conversation analysis model to check whether the report interpretation intention exists during the conversation process comprises:
invoking the conversation analysis model based on each piece of instant conversation content to generate a model output result;
in response to the model output result representing that the report interpretation intention exists, performing keyword matching on each piece of instant conversation content using a second matching rule;
in response to the matching being successful, concluding that the report interpretation intention exists during the conversation process; and
in response to the matching failing, concluding that the report interpretation intention does not exist during the conversation process.

6. The method of claim 2, wherein the generating a model prompt based on the respective field contents comprises:
generating the model prompt based on the respective field contents and each piece of instant conversation content.

7. The method of claim 1, wherein the obtaining respective field contents comprised in the first image in response to the first image belonging to a medical examination report comprises:
performing image recognition and information extraction on the first image to obtain the respective field contents comprised in the first image;
performing keyword matching on the respective field contents using a third matching rule; and
concluding that the first image belongs to the medical examination report in response to the matching being successful.

8. The method of claim 1, wherein the obtaining respective field contents comprised in the first image in response to the first image belonging to a medical examination report comprises:
inputting the first image into a preset classification model, and obtaining a classification probability of the first image belonging to an image category corresponding to the medical examination report;
concluding that the first image belongs to the medical examination report in response to the classification probability exceeding a preset threshold; and
performing image recognition and information extraction on the first image to obtain the respective field contents comprised in the first image.

9. The method of claim 8, wherein after the performing image recognition and information extraction on the first image to obtain the respective field contents comprised in the first image, the method further comprises:
performing keyword matching on the respective field contents using a fourth matching rule;
concluding that the first image belongs to the medical examination report in response to the matching being successful; and
concluding that the first image does not belong to the medical examination report in response to the matching failing.

10. The method of claim 1, wherein the using a received image as a first image in response to an image uploading operation comprises:
displaying an image uploading interface in response to a triggering operation on a report interpretation control, and displaying an image uploading control in the image uploading interface; and
using the received image as the first image in response to a triggering operation on the image uploading control.

11. An electronic device, comprising:
a processor;
a memory, configured to store executable instructions; and
wherein the processor is configured to read the executable instructions from the memory, and execute the executable instructions to implement a content generation method, and the content generation method comprises:
using a received image as a first image in response to an image uploading operation;
obtaining respective field contents comprised in the first image in response to the first image belonging to a medical examination report; and
generating a model prompt based on the respective field contents, and invoking a report interpretation model based on the model prompt to generate a report interpretation result of the first image; wherein the report interpretation model is obtained by fine-tuning a pre-trained generative model using medical examination report samples in advance.

12. The electronic device of claim 11, wherein the using a received image as a first image in response to an image uploading operation comprises:
displaying a conversation interface in response to a start operation of a conversation function, and receiving instant conversation content through the conversation interface;
analyzing the instant conversation content to check whether a report interpretation intention exists during a conversation process;
in response to the report interpretation intention existing during a conversation process, displaying prompt conversation content of a medical report interpretation function in the conversation interface, the prompt conversation content comprising a function entry control;
displaying an image uploading interface in response to a triggering operation on the function entry control, and displaying an image uploading control in the image uploading interface; and
using the received image as the first image in response to a triggering operation on the image uploading control.

13. The electronic device of claim 12, wherein the analyzing the instant conversation content to check whether a report interpretation intention exists during a conversation process comprises:
analyzing the instant conversation content using a fifth matching rule and/or a conversation analysis model to check whether the report interpretation intention exists during the conversation process, the conversation analysis model being a pre-trained language model.

14. The electronic device of claim 13, wherein the analyzing the instant conversation content using a fifth matching rule and a conversation analysis model to check whether the report interpretation intention exists during the conversation process comprises:
performing keyword matching on each piece of instant conversation content using a first matching rule; and
in response to the matching being successful, invoking the conversation analysis model based on first content to generate a model output result, the model output result representing whether the report interpretation intention exists during the conversation process, the first content comprising at least one selected from the group consisting of each piece of instant conversation content, a keyword that is successfully matched, and the instant conversation content corresponding to the keyword.

15. The electronic device of claim 13, wherein the analyzing the instant conversation content using a fifth matching rule and a conversation analysis model to check whether the report interpretation intention exists during the conversation process comprises:
invoking the conversation analysis model based on each piece of instant conversation content to generate a model output result;
in response to the model output result representing that the report interpretation intention exists, performing keyword matching on each piece of instant conversation content using a second matching rule;
in response to the matching being successful, concluding that the report interpretation intention exists during the conversation process; and
in response to the matching failing, concluding that the report interpretation intention does not exist during the conversation process.

16. The electronic device of claim 12, wherein the generating a model prompt based on the respective field contents comprises:
generating the model prompt based on the respective field contents and each piece of instant conversation content.

17. The electronic device of claim 11, wherein the obtaining respective field contents comprised in the first image in response to the first image belonging to a medical examination report comprises:
performing image recognition and information extraction on the first image to obtain the respective field contents comprised in the first image;
performing keyword matching on the respective field contents using a third matching rule; and
concluding that the first image belongs to the medical examination report in response to the matching being successful.

18. The electronic device of claim 11, wherein the obtaining respective field contents comprised in the first image in response to the first image belonging to a medical examination report comprises:
inputting the first image into a preset classification model, and obtaining a classification probability of the first image belonging to an image category corresponding to the medical examination report;
concluding that the first image belongs to the medical examination report in response to the classification probability exceeding a preset threshold; and
performing image recognition and information extraction on the first image to obtain the respective field contents comprised in the first image.

19. The electronic device of claim 18, wherein after the performing image recognition and information extraction on the first image to obtain the respective field contents comprised in the first image, the method further comprises:
performing keyword matching on the respective field contents using a fourth matching rule;
concluding that the first image belongs to the medical examination report in response to the matching being successful; and
concluding that the first image does not belong to the medical examination report in response to the matching failing.

20. A non-transitory computer-readable storage medium, wherein the non-transitory computer-readable storage medium stores a computer program, the computer program is executed by a processor to cause the processor to implement a content generation method, and the content generation method comprises:
using a received image as a first image in response to an image uploading operation;
obtaining respective field contents comprised in the first image in response to the first image belonging to a medical examination report; and
generating a model prompt based on the respective field contents, and invoking a report interpretation model based on the model prompt to generate a report interpretation result of the first image; wherein the report interpretation model is obtained by fine-tuning a pre-trained generative model using medical examination report samples in advance.
